(19) United States
(12) Patent Application Publication
(10) Pub. No.: US 2004/0185474 A1
(43) Pub. Date: Sep. 23, 2004

(54) Method of diagnosing depression

(76) Inventors: Kazuhito Rokutan, Osaka (JP); Tetsuro Ohmori, Tokushima (JP); Toshiro Saito, Hatoyama (JP); Hiroyuki Tomita, Kawasaki (JP); Hirokazu Kato, Hatoyama (JP); Masatoshi Narahara, Sayama (JP)

Correspondence Address: ANTONELLI, TERRY, STOUT & KRAUS, LLP, 1300 NORTH SEVENTEENTH STREET, SUITE 1800, ARLINGTON, VA, 22209-9889, US

(21) Appl. No.: 10/739,329

(22) Filed: Dec. 19, 2003

(30) Foreign Application Priority Data
Dec. 27, 2002 (JP) 2002-380614

(51) Int. Cl. C12Q 1/68

(52) U.S. Cl. 435/6

(57) ABSTRACT

This invention relates to a method of simply and accurately diagnosing depression by assaying the expression levels of a specific group of genes in a subject's peripheral leukocytes. In this method, the expression level of at least one gene selected from among apoptosis-associated genes, ATPase-associated genes, cell cycle-associated genes, cytokine-associated genes, heat shock protein-associated genes, polymerase-associated genes, GTP-binding protein-associated genes, protein kinase C-associated genes, and mitochondrial cytochrome C oxidase-associated genes is analyzed using mRNA of a subject's peripheral blood, and symptoms of depression in the subject are diagnosed based on the results of such analysis.

This patent application claims priority from Japanese Patent Application No. 2002-380614 filed on Dec. 27, 2002, and includes part or all of the contents as disclosed in the description thereof.

### TECHNICAL FIELD

The present invention relates to a method of diagnosing depression.

### BACKGROUND ART

Depression is a disease with high lifetime morbidity of approximately up to 10%, and this rate is predicted to further increase in the future due to stress in contemporary society. This disease seriously afflicts patients mentally and physically and imposes enormous damage upon their social lives. In addition, it is a serious disease that often leads to suicide. It is deduced that many of the people who commit suicide (as many as 30,000 or more per year) are afflicted with depression. This disease is also deeply associated with societal problems such as truancy, unemployment, and social withdrawal or medical problems such as alcohol-related disorders. Establishment of mechanisms for precisely diagnosing and promptly treating this disease is indispensable for improving the lives of people, and thus is an urgent need of society as a whole.

Diagnosis of depression is, however, far from simple. Cardinal symptoms of depression are, for example, depressive mood, hypobulia, loss of interest and pleasure, disrupted concentration and attention, lowered self-esteem and self-confidence, feelings of guilt and worthlessness, pessimism about the future, thoughts of suicide, sleep disorders, and loss of appetite. These symptoms have features peculiar to depression, which differ from depressed feelings experienced by anyone, and also differ from the lowered mental activity and sense of exhaustion experienced by people afflicted with physical diseases. The symptoms of depression are mainly comprehended by taking a precise medical history, questioning when and how the symptoms in terms of mental activity were developed and what types of damages have been imposed upon their social and domestic lives, and confirming various symptoms based on a patient's attitude or the contents of conversations during consultation. For example, family medical history, anamnesis; physical conditions, early developmental history, life history, personality inclination, premorbid social adaptation, and the occurrence of any episode(s) that had triggered the disease can be important references. In order to accurately comprehend these factors, an interview needs to be conducted by a highly skilled specialist in psychiatric medicine for approximately 1 hour. Further, it should be confirmed that a patient does not have any major abnormalities in terms of general physical or neurological conditions. If necessary, the possibility of the existence of organic brain disorders is to be eliminated by electroencephalography or brain imaging tests. The patient is then subjected to diagnosis. The findings are compared with the diagnostic standards issued by the World Health Organization (WHO) or the American Psychiatric Association, and the diagnosis can be generally confirmed.

As a major drawback, conventional diagnostic methods require skilled techniques. Needless to say, thorough knowledge and practice concerning depression are required. However, there are numerous psychological, mental, and physical states that result in the exhibition of depressive conditions even though they are not forms of depression. Differential diagnosis also becomes essential. Accordingly, diagnosis must be conducted by a thoroughly trained specialist in psychiatric medicine. Depression, which is a common disease with lifetime morbidity of approximately 10%, however, is often the subject of consultation with primary care doctors. Diagnosis of depression without objective medical findings is not always easy for general doctors who may not be acquainted with psychiatric consultation. Depression is a medical disease that requires treatment of the body (brain), including medication. Accordingly, it is difficult for specialists in clinical psychology, such as clinical psychotherapists, or mental health workers, such as public health nurses, to independently diagnose depression.

Technical skill is required for diagnosis mainly because of a lack of simple and objective methods of diagnosis regarding symptoms. Although there is a screening method utilizing a self-administered questionnaire, people tend to fill in the questionnaire based on their subjective viewpoints. Thus, genuine depression cannot be distinguished from depressed feelings caused by personality-based factors, environmental factors, or poor physical conditions. Symptom rating scales employed by doctors are often used in determination of severity, although adequate questioning is required to evaluate each item. Thus, such methods cannot be alternatives to diagnosis.

Many testing methods have been heretofore attempted, with the aim of utilizing them as objective indicators. Depression causes functional alteration in brain monoamine systems. This alteration is known to have a considerable influence upon the neuroendocrine system, the neuroimmune system, and the autonomic nervous system via psychosomatic correlation. In particular, the application of the results of a dexamethasone suppression test that allows accurate comprehension of neuroendocrine abnormalities, i.e., a minor level of adrenal cortical hormone hypersecretion, to diagnosis of depression has been extensively examined from the 1980s onwards. Clinical application thereof was, however, not realized due to the necessity for complicated procedures such as the administration of test drugs and limitations in terms of sensitivity or specificity. At the study phase, other abnormalities in the neuroendocrine system, the neuroimmune system, the autonomic nervous system, circadian rhythms, sleep architecture, and the like had been reported. Recently, changes regarding conditions of brain blood flow or brain monoamine receptors are also pointed out as objective indicators, although they are still disadvantageous in terms of sensitivity and reproducibility. Given the aforementioned factors, diagnosis of a complicated mental disease, i.e., depression, is difficult by a method of testing limited factors. Enormous amounts of time and labor are required to perform conventional testing methods and to diagnose the disease. From the viewpoint of simplicity, conventional techniques cannot be applied to routine medical care at present.

In the past, the catecholamine hypothesis, the indoleamine hypothesis, the GABA hypothesis, the glutamine hypothesis, the dopamine hypothesis, the neurogenesis hypothesis, and the like have been proposed as causes of depression. Many discrepancies of these hypotheses have been pointed out, and they have not yet resulted in conclusions. Linkage studies and association studies based on molecular genetic engineering and the search for sensitive domains of chromosomes by linkage analysis have been carried out. In the case of a disease such as depression, the diathesis (biological feature) of which is generated through interactions among multiple genes and environmental factors such as stress, analysis of the pathogenic gene is extremely difficult. Based on past gene analysis, genes such as those related to serotonin transporter, serotonin 1A/2 C receptor, dopamine D2/D3 receptor, dopamine transporter, tyrosine hydroxylase, tryptophan hydroxylase, monoamine oxidase, and ATPase have been reported as candidate functional genes associated with depression. For example, the correlation between Na/K-ATPase and mental diseases, such as depression (Depress Anxiety 1997, 5, pp. 53-65) or dysthymia (J. Basic Clin. Physiol. Pharmacol. 2000, 11 (4), pp. 375-94), has been pointed out. Improvement of symptoms caused by an antidepressant, i.e., carbamazepine, is reported to be correlated with elevation of erythrocyte Na/K-ATPase activity (Neuropsychobiology 1999, 40 (3), pp. 134-9). Some researchers are, however, skeptical about the aforementioned reports, and additional tests have been conducted thereon.

### SUMMARY OF THE INVENTION

An object of the present invention is to provide a method of diagnosing depression with high accuracy by measuring many factors in a simple manner.

The present inventors have focused on peripheral leukocytes that can be easily obtained as specimens and allow many receptors of factors associated with stress responses to be expressed therein in order to objectively evaluate the conditions of depression, in the development of which stress plays an important role. They have extensively analyzed the expression patterns of mRNAs of 1,500 genes associated with stress responses and then developed certain patterns. Thus, they have found a method that is capable of diagnosing depression with high accuracy. This has led to the completion of the present invention.

More specifically, the present invention relates to a method of diagnosing depression, wherein the expression level of at least one gene selected from among apoptosis-associated genes, ATPase-associated genes, cell cycle-associated genes, cytokine-associated genes, heat shock protein-associated genes, polymerase-associated genes, GTP-binding protein-associated genes, protein kinase C-associated genes, and mitochondrial cytochrome C oxidase-associated genes is analyzed using mRNA of a subject's peripheral blood, and symptoms of the subject are diagnosed based on the results of the aforementioned analysis.

The present invention also relates to a method of diagnosing depression, wherein the expression level of at least one gene selected from among the genes listed in Table 1 is analyzed using mRNA of a subject's peripheral blood, and symptoms of the subject are diagnosed based on the results of the aforementioned analysis. Diagnosis is preferably carried out by analyzing gene expression profiles. Analysis can be carried out with, for example, the cluster analysis technique.

In an embodiment of the present invention, the method of diagnosing depression is characterized in that the gene expression profiles of a subject and that of a healthy individual are compared, and the results of the comparison are analyzed, thereby allowing diagnosis regarding symptoms of the subject.

In another embodiment of the present invention, the method of diagnosing depression is characterized in that the gene expression profiles of a single subject before and after treatment are compared, and the results of the comparison are analyzed, thereby allowing diagnosis regarding symptoms of the subject.

In the method of the present invention, the gene expression level is analyzed by any technique: for example, nucleic acid hybridization utilizing DNA-immobilized samples such as DNA chips, DNA arrays, or membrane filters; quantitative PCR such as RT-PCR or real-time PCR; Northern blotting; subtraction; differential display; differential hybridization; or cross-hybridization. A technique utilizing DNA chips is particularly preferable.

In the method of the present invention, mRNA derived from peripheral blood is preferably derived from peripheral leukocytes.

The present invention also relates to a solid substrate for diagnosing depression comprising a probe, immobilized on the substrate, that specifically hybridizes to at least one gene selected from among the genes listed in Table 1 to detect the selected gene.

The present invention further provides a system for diagnosing depression that utilizes the method of diagnosing depression of the present invention. This system diagnoses depression by comparing the data concerning a subject, such as gene expression level, age, and sex, with data that had been previously acquired concerning a healthy individual, and then analyzing the results of the comparison.

### BRIEF DESCRIPTION OF THE DRAWINGS

FIG. 1 shows the results of gene expression analysis in each sample.

FIG. 2 shows the results of gene expression analysis in each sample.

FIG. 3 shows the results of gene expression analysis in each sample.

FIG. 4 shows the results of gene expression analysis in each sample.

FIG. 5 shows the results of gene expression analysis in each sample.

FIG. 6 shows the results of gene expression analysis in each sample.

FIG. 7 shows the results of gene expression analysis in each sample.

FIG. 8 shows the results of gene expression analysis in each sample.

FIG. 9 shows the results of gene expression analysis in each sample.

FIG. 10 shows the results of gene expression analysis in each sample.

FIG. 11 schematically shows the method of diagnosing depression of the present invention, wherein F1 indicates a DNA chip, F2 indicates probe DNA corresponding to the gene selected in the present invention, F3 indicates an excitation light source and a fluorescence detector, and F4 indicates a computer for controlling the fluorescence detector.

FIG. 12 schematically shows the system for diagnosing depression of the present invention, wherein the personal information database stores information concerning sex, age, and the like.

### DETAILED DESCRIPTION OF THE INVENTION

The present inventors collected blood from patients and healthy volunteers as described below. RNA was extracted from the whole blood, and gene expression of patients was then analyzed using DNA chips, along with that of healthy volunteers. A DNA chip comprises DNA fragments having nucleotide sequences corresponding to numerous genes immobilized on a substrate such as a glass substrate, and it is used for detecting DNA or RNA in a sample by hybridization. Instead of the aforementioned DNA chip, other solid substrates comprising DNA fragments to be analyzed immobilized thereon, for example, DNA arrays or membrane filters, can be adequately used.

Target patients were as follows. Target patients were those who had agreed with the written description for participating in the research for developing the present diagnostic method selected from among untreated patients afflicted with depression who had visited the Department of Psychiatry and Neurology of the Tokushima University Hospital between November 2001 and June 2002. This research was approved by the ethics committee of Tokushima University Hospital. Diagnosis was made in accordance with depressive episode of specified in the International Classification of Diseases, 10th revision (ICD-10). Patients with serious physical complications or those taking therapeutic agents for physical diseases were excluded. Blood was collected by a doctor or nurse between 10:00 am and 1:00 pm from the patients under fasting conditions through cubitus veins under resting conditions.

Fourteen patients whose samples before treatment had been obtained were 11 males and 3 females aged 28 to 70 (46 years old on average), and their Hamilton scores were between 12 and 34 points (23.7 points on average).

The mRNA expression levels of these patients were compared with those of healthy controls with the same sex and age conditions. Blood was collected from healthy volunteers under fasting conditions between 10:00 am and 1:00 pm.

Samples during symptom-free periods after the treatment had been obtained from 6 patients. They were 5 males and 1 female aged 31 to 71 (49.8 years old on average), and their Hamilton scores were between 1 and 9 (5.5 on average). Treatment was mainly carried out by drug therapy using antidepressants. The remission of symptoms was determined based on general clinical diagnosis. Samples satisfied the standard of having scores of 7 or less on the Hamilton Rating Scale, which are generally regarded as representing remission of symptoms, except for one sample. Samples after treatment were collected 68 to 211 days after the collection of samples before treatment (121 days on average). The mRNA expression level after treatment was compared with that of a sample taken before treatment from the same subject.

Blood (5 ml) was collected from each of the 14 patients whose samples before treatment had been obtained, and total RNA was extracted using a PAXgene Blood RNA System (Qiagen). The yield of total RNA was 5 μg to 15 μg. Subsequently, 5 μg of total RNA extracted from each patient was separated, annealed with an oligo (dT) 24 primer comprising a T7 promoter sequence added thereto, and first-strand DNA was synthesized. Thereafter, this first-strand DNA was used as a template to synthesize second-strand DNA having a T7 promoter sequence. Finally, the second-strand DNA was used as a template to synthesize RNA with the aid of T7 RNA polymerase. A random hexamer was annealed to 6 μg of the synthesized RNA to conduct a reverse transcriptase reaction, and Cy5-dCTP was incorporated into the strand. Thus, fluorescence-labeled cDNA was synthesized. In a manner similar to the case of these patients, 5 ml of blood was collected from each of 14 healthy volunteers with the same sex and age conditions, and total RNA was then extracted. cDNA was similarly synthesized except for the use of Cy3 as a fluorescent label.

When comparing samples of a single subject before and after treatment, cDNA labeled with Cy5 and cDNA labeled with Cy3 were synthesized from the samples before and after treatment respectively.

Equivalent amounts of two types of cDNAs for comparison and analysis were mixed, the resultant was applied to a DNA chip (a DNA chip for analyzing drug response, Hitachi Co., Ltd.), and hybridization was carried out at 62° C. for 12 hours. After washing, fluorescence intensity at each spot was assayed using a scanner (ScanArray 5000, GSI-Lumonics). Differences in gene expression levels between samples obtained from patients and samples obtained from healthy controls or those between samples obtained from a single patient before and after treatment were determined.

Analysis was carried out as follows. A group of genes (692 genes) having fluorescence intensities of 500 or higher in all 20 groups was selected and subjected to cluster analysis for classifying the group of genes. Analysis was carried out by aggregative clustering based on the average Euclidean distance without a weight between clusters.

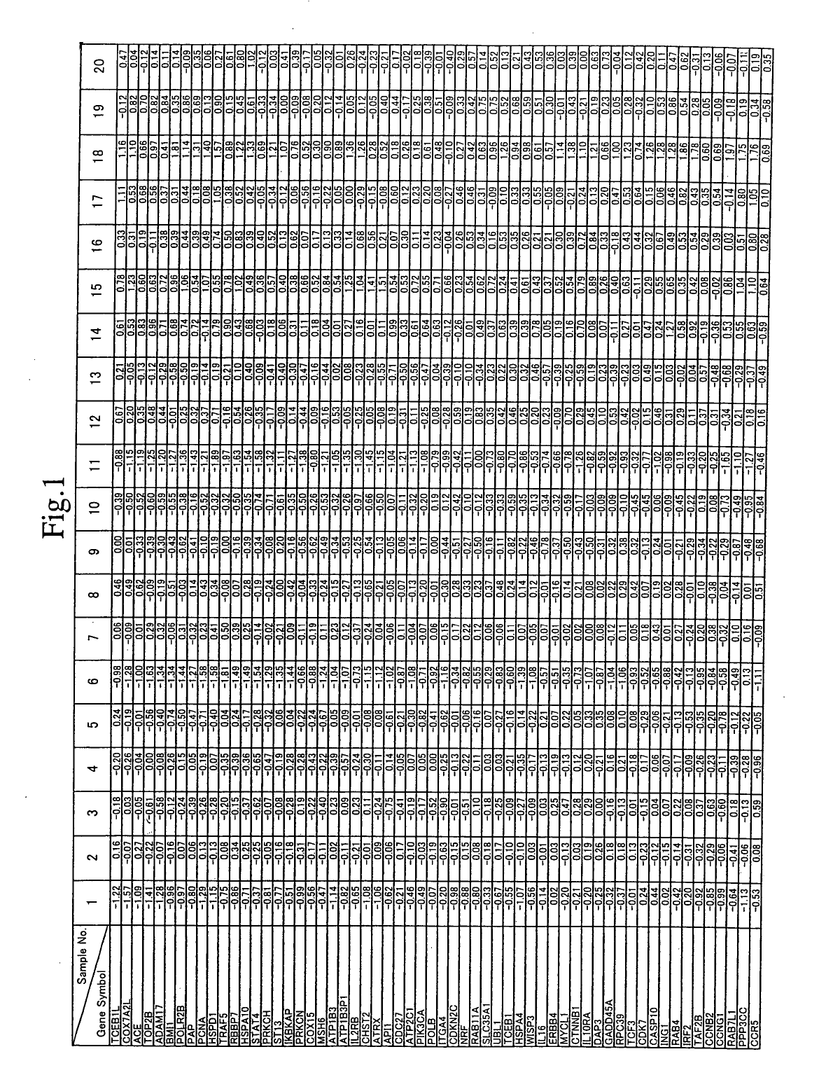

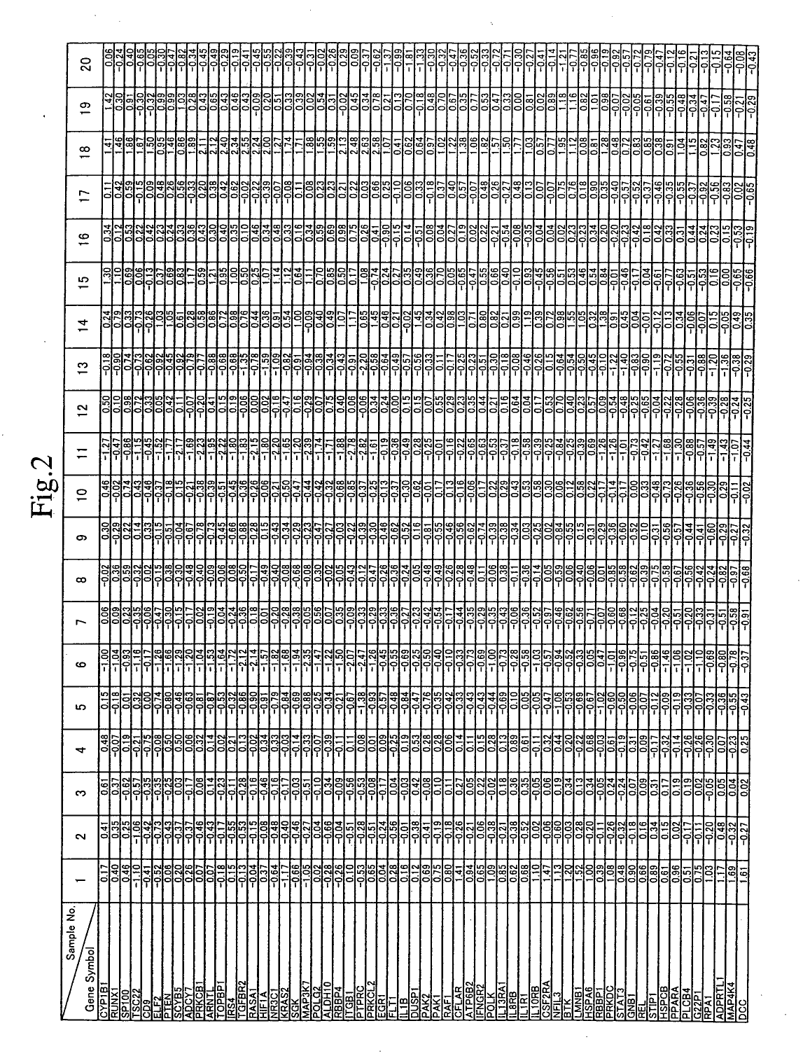

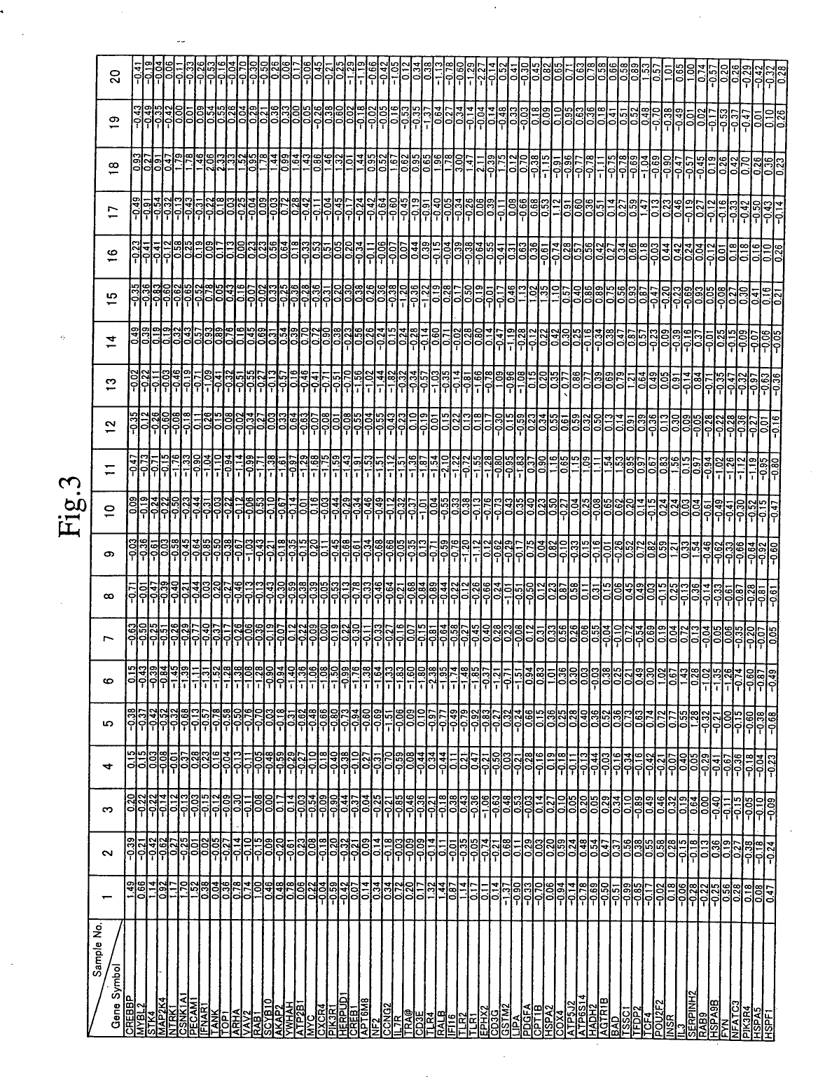

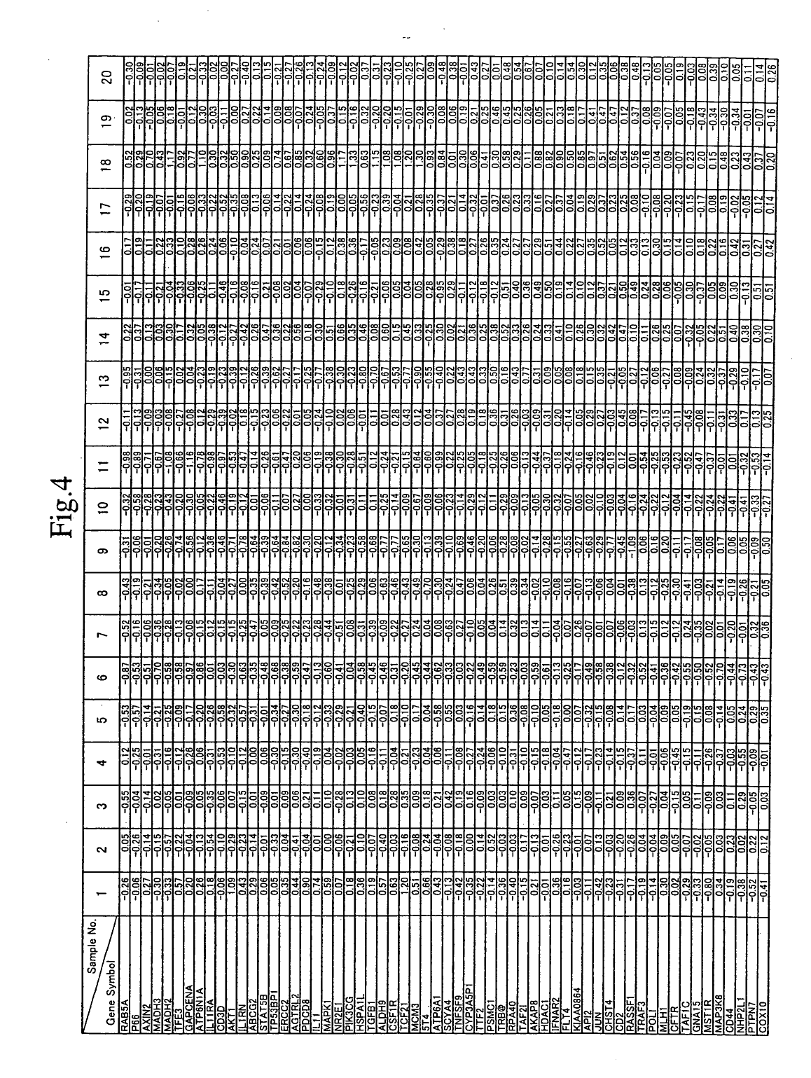

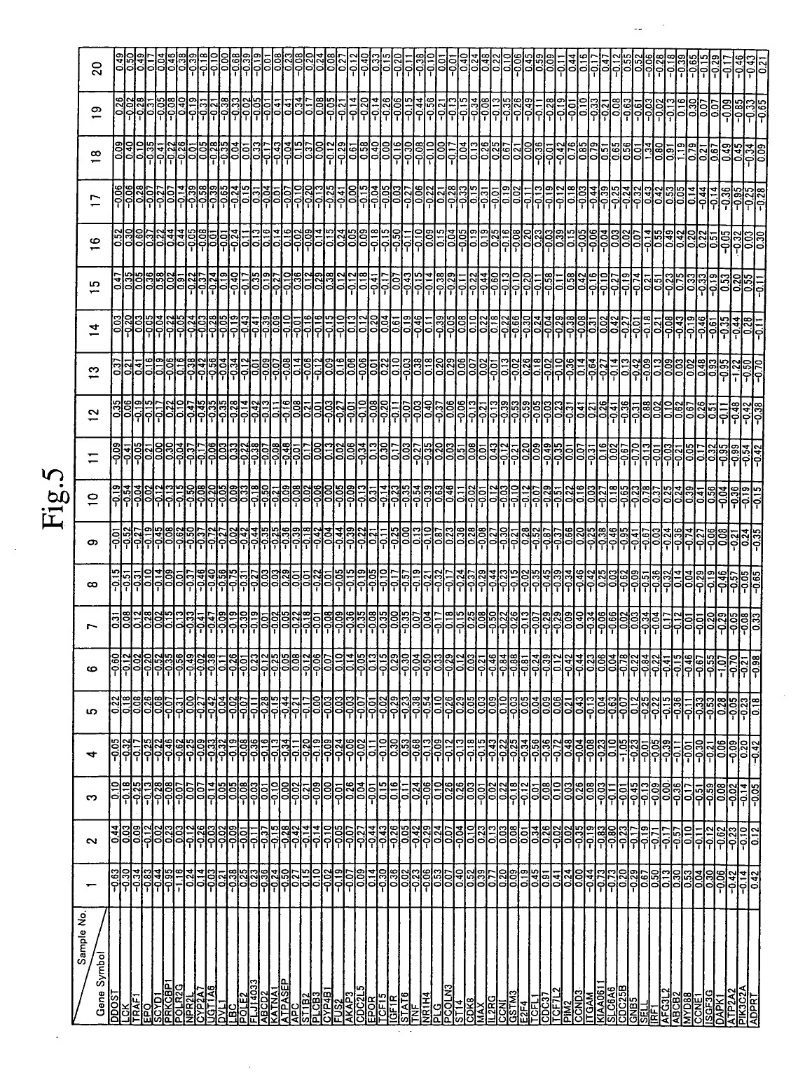

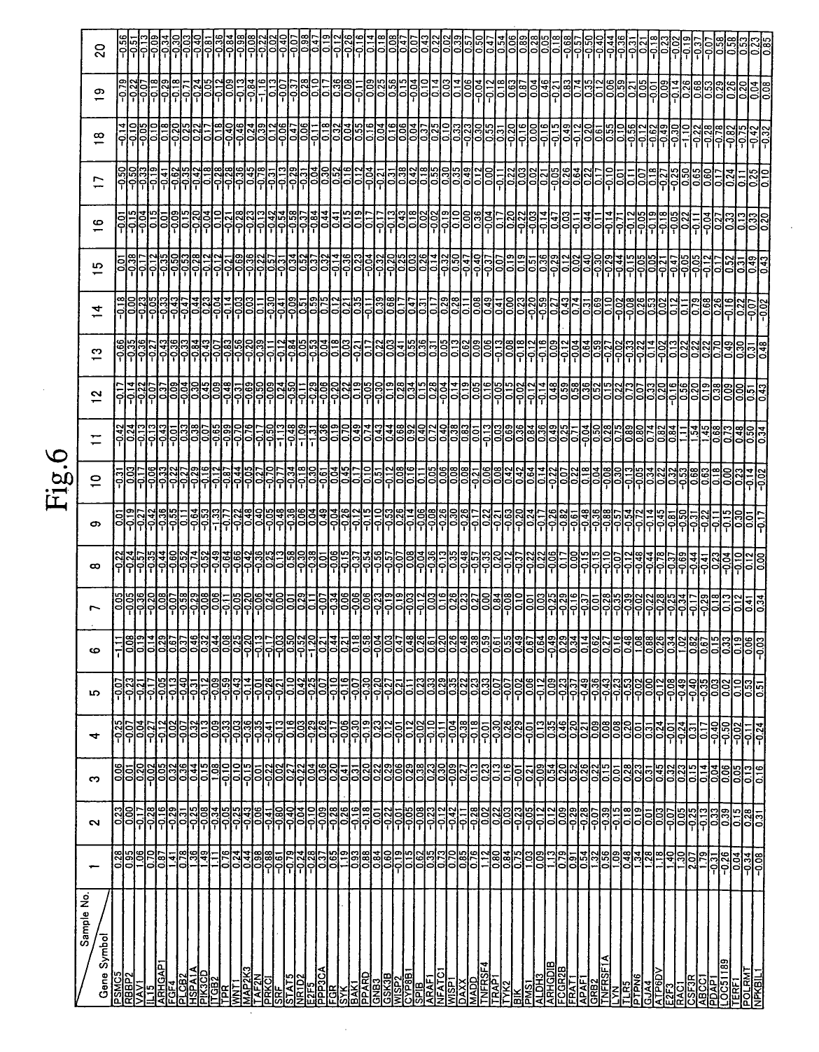

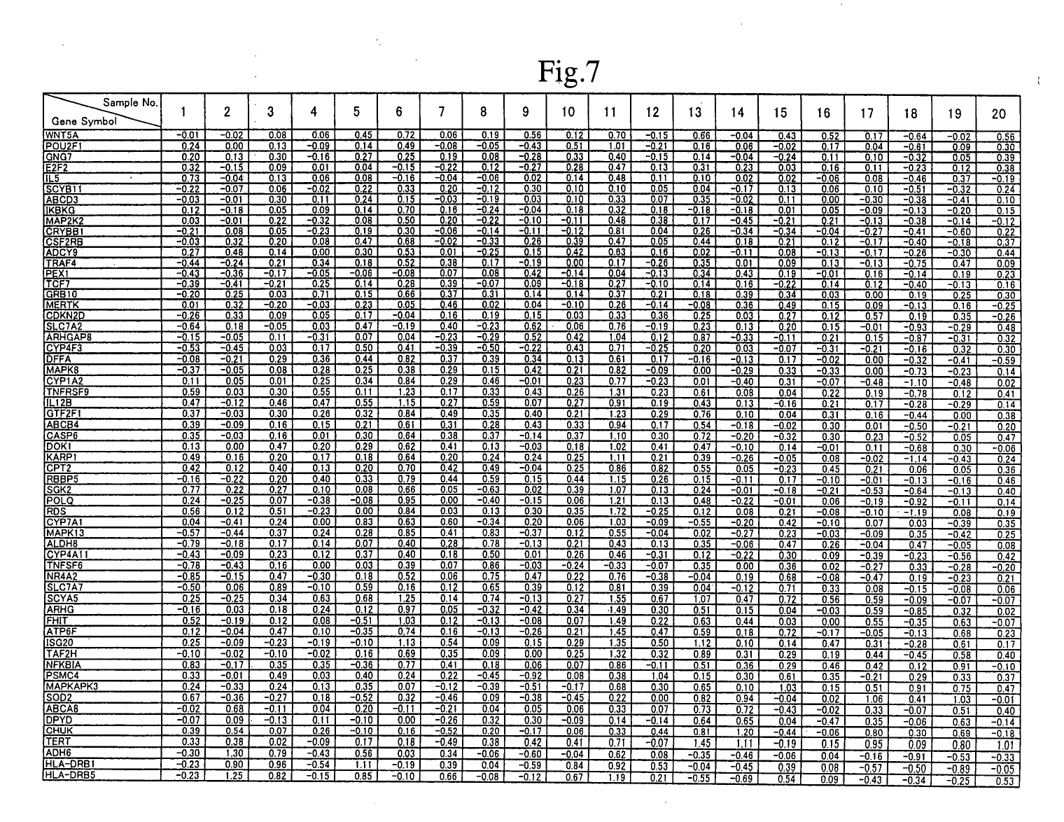

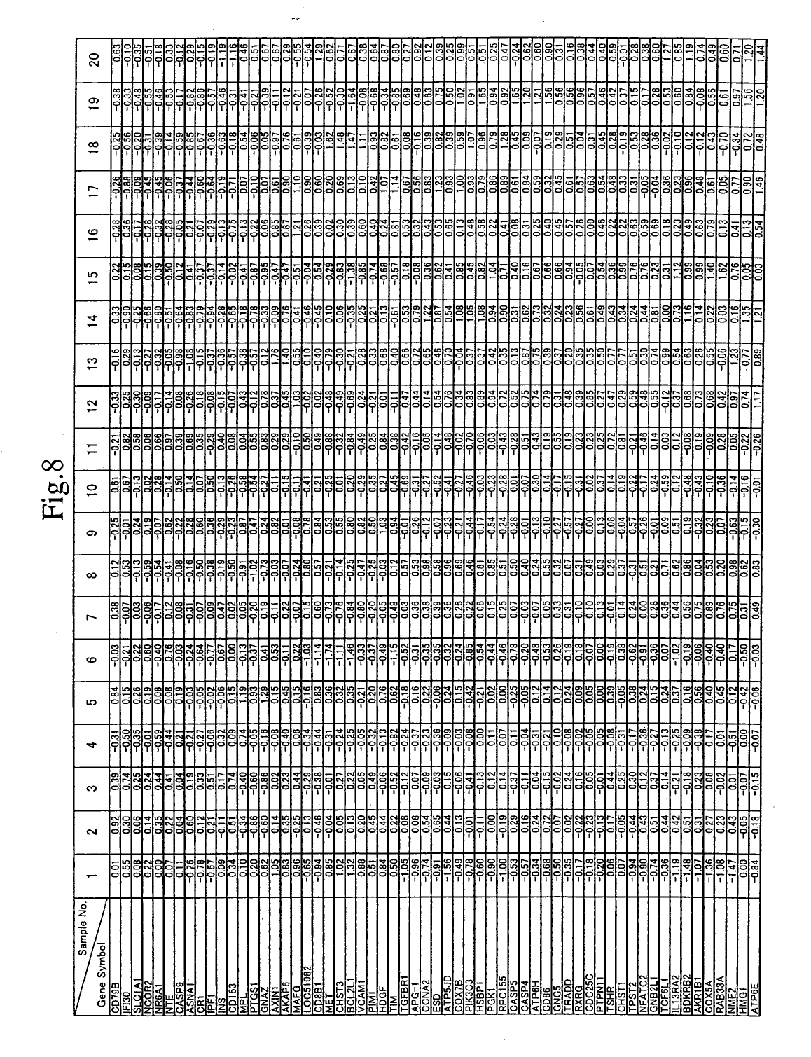

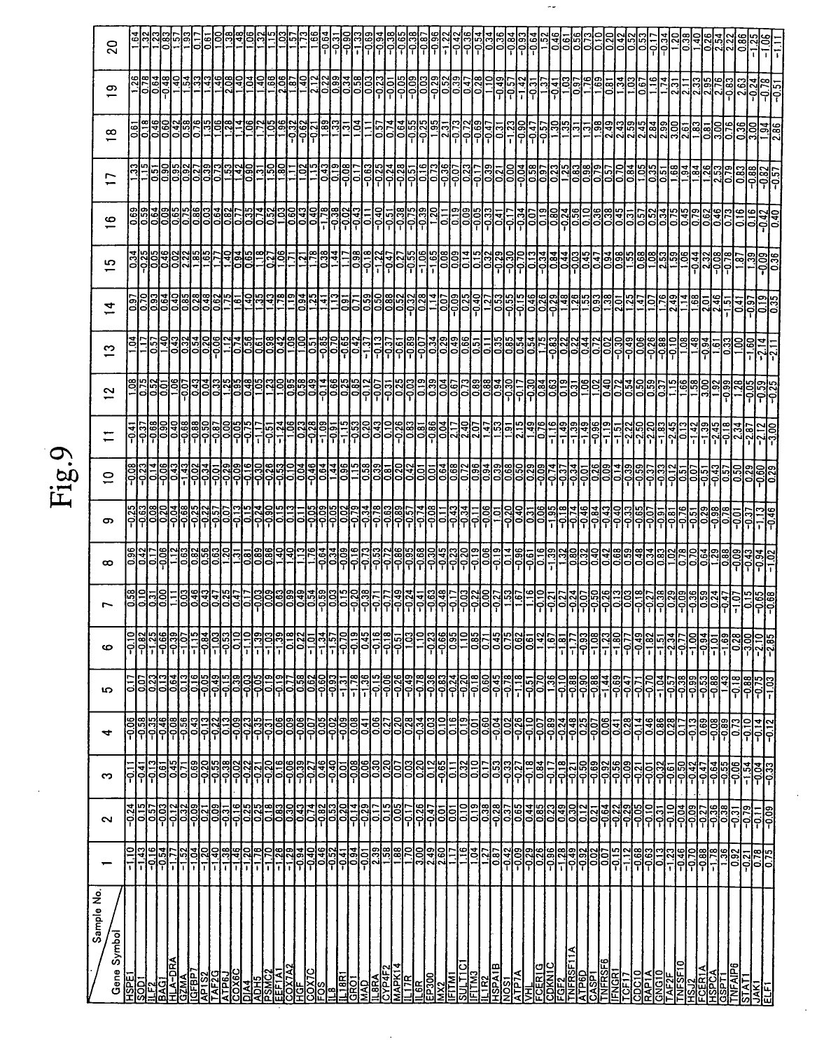

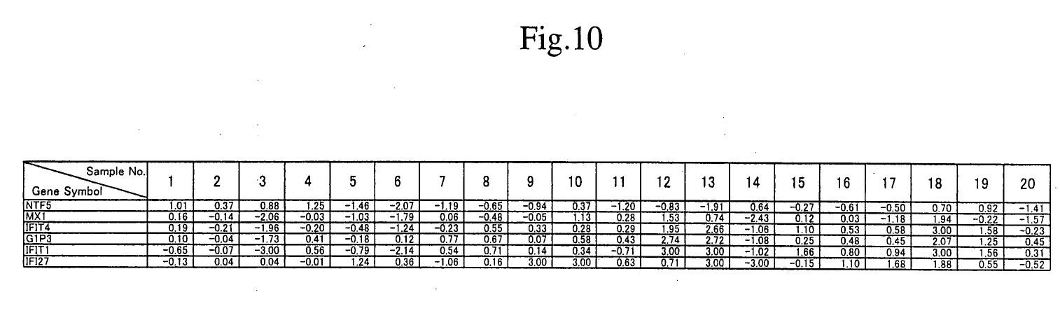

The results of analysis are shown in FIGS. 1 to 10. In the drawings, the logarithms (base: 2) of the gene expression ratio (Cy5/Cy3) are shown. Samples Nos. 1 to 14 are comparisons of patients (Cy5) and healthy controls (Cy3), and Samples Nos. 15 to 20 are comparisons of a single patient before treatment (Cy3) and after treatment (Cy5). As is apparent from FIGS. 1 and 2, the expression level of a specific group of genes (TCEBlL to CCR5 in FIG. 1, CYP1B1 to PTPRC in FIG. 2) are decreased in the patient group compared to that in the healthy control group (Samples Nos. 1 to 14). At the same time, the expression levels are significantly elevated after treatment compared with those before treatment (Samples Nos. 15 to 20). The results of analysis are easily conceived if logarithmic values for the expression ratio (Cy5/Cy3) are represented by different colors depending on, for example, specified ranges of numerical values. In these groups of genes, expression levels are lower in an afflicted state than in a healthy state. When the symptoms have improved after treatment, the expression level is elevated compared with that before treatment. Accordingly, these groups of genes were found to be useful as diagnostic markers for depression. More specifically, when the expression levels of these genes of a subject are lower than those of healthy individual or those in a healthy state, it can be determined that the subject is highly likely to be afflicted with depression. Regarding other genes, no significant difference was observed between Samples Nos. 1 to 14 and Samples Nos. 15 to 20.

Groups of genes selected as diagnostic markers for depression are collectively shown in Table 1. Table 1 demonstrates that many apoptosis-associated genes, ATPase-associated genes, cell cycle-associated genes, cytokine-associated genes, heat shock protein-associated genes, polymerase-associated genes, GTP-binding protein-associated genes, protein kinase C-associated genes, and mitochondrial stress-responsive cytochrome C oxidase-associated genes are included among the selected genes. This reveals that depression can be diagnosed by assaying the expression levels of the genes included in the above categories.

At present, mechanisms of depression are indefinite, although the following is known as a correlation between the group of genes selected as a diagnostic marker and depression.

The angiotensin-converting enzyme (ACE) serves as the key to regulating the renin-angiotensin system and also regulates the dopamine turnover in the midbrain. Accordingly, the association thereof with mental diseases has been identified as a candidate for sporadic Alzheimer's disease-associated genes (Eur J Hum Genet. 2001: 9(6): 437-444). Association of the ACE gene polymorphism with schizophrenia has also been analyzed (Neuropsychobiology 2001; 44(1): 31-35).

Recently, the concept of perceiving clinical conditions involved with ion channel dysfunctions as “channel diseases” has been proposed. An ion channel serves as the most important function for neuron cell activity, and its association with epilepsy, ataxia, migraine, schizophrenia, Alzheimer's disease, and other neurodegenerative diseases has been pointed out (CNS Drug Rev 2001; 7(2): 214-240). Concerning Na/K-ATPase and mental diseases, association of the ion channel with depression (Depress Anxiety 1997, 5, pp. 53-65) or dysthymia (J. Basic Clin. Physiol. Pharmacol. 2000, 11 (4), pp. 375-94) has been particularly noted. For example, the association between the Na/K-ATPase α subunit ATP1A3 (Biol Psychiatry 1998; 44: 47-51) or β subunit ATP1B3 (Biol Psychiatry 1995; 37: 235-244) and bipolar disorders has been reported. Further, improvement of symptoms caused by an antidepressant, carbamazepine, is known to be correlated with elevation of erythrocyte Na/K-ATPas e activity (Neuropsychobiology 1999, 40 (3), pp. 134-9). ATP1B3P1 is a pseudogene of ATP1B3 and is transcribed from the same genome. ATPX is ATPase that was discovered in 1995, and association thereof with X-chromosome-linked mental retardation (e.g., ATR-X syndrome, Carpenter syndrome, Juberg-Marsidi syndrome, or Smith-Fineman-Myers syndrome) has been pointed out. ATP2C1 is a novel calcium pump (P-type Ca-transporting ATPase), and its mutation is deduced to be a cause of the Hailey-Hailey disease. The present inventors have verified for the first time that variations in expression patterns of ATPase mRNA including its isozymes would reflect the state of depression.

Inflammatory cytokines such as interleukins (IL)-1, 6, and 8 are associated with stress responses, and affect the central nervous system, thereby causing drowsiness, loss of appetite, and other symptoms. As a major side effect of interferon a used for treating hepatitis C, development of depression is known.

According to the analysis of patients afflicted with depression conducted by the present inventors, mRNA expression levels of, for example, cytokine receptors (CRF2-4; CRFB4), IL-12 β2 receptors, IL-13 receptor third chain, type-1 IL-1 receptor homolog (IL-1Rrp), IL-2 receptor β chain, IL-4 receptor, IL-7 receptor, IL-8, interferon regulatory factor 4 (LSIRF/IRF4), interferon-induced protein (ISG15), interferon y-induced protein IFI16, interferon a-responsive gene IFI127, novel interferon-responsive genes IFIT1 and IFIT4, interferon-induced mRNA fragment (G1P3), interferon α receptor, interferon γ receptor, novel cytokines (GR01, GR02), platelet-derived chemokine (SCYB5), CC chemokine receptor (CCR5), T cell receptors (CD3E, CD3G, CD3Z, CD8B1), and IgE receptor α chain were lowered. Also, lowered mRNA expression levels of, for example, IkB kinase scaffold protein (IKBKAP), JAK1, and MAP kinase family (MAPK2k1, MAP3K1, MAP3K7, MAP4K4) associated with cytokine signal transduction, were observed. In contrast, mRNA expression levels of GM-CSF receptor, IF127, interferon-responsive genes (IFITM1, IFITM3), interferon α, IL-10 receptor subunit (IL10RB), NK cell stimulatory factor (IL-12B), IL-17 receptor, IL-1 receptor antagonist, IL-3 and IL-6 receptors, IL-8 and IL-8α receptors, interferon-induced proteins (ISG15, ISG20, MX1, and MX2), macrophage inflammatory protein (GOS19-1), RANTES, and TRAIL receptor 3 were elevated. In the case of patients with stress-induced depression, expression levels of many cytokine-associated genes were varied. Variation in the expression levels of interferon-associated genes was particularly high, and it would be data that demonstrate the association between the interferon therapy and depression.

Further, 14 patients afflicted with depression and healthy volunteers were compared, expression patterns before and after the remission of symptoms were analyzed, and gene expression patterns peculiar to depression were evaluated by cluster analysis. As a result, mRNAs of TNF-α convertase enzyme (ADAM17), IL-16, IL-10 receptor, interferon regulatory factor 2, tumor necrosis factor receptor-associated factor 5 (TRAF5), IL-2 receptor β chain, and platelet-derived chemokine (SCYB5) of patients afflicted with depression were all lowered, and expression levels were enhanced after treatment. Expression levels of NF-kappa B-repressing factors (NRF) binding to the suppressor motif of the IL-8 gene promoter that had been newly discovered were lowered commonly among them, and its association with lowered expression levels of many inflammatory cytokines has drawn attention. Furthermore, the expression level of CD45 mRNA, a representative T lymphocyte receptor tyrosine phosphatase, is lowered by depression, and the expression level was restored as the symptoms improved. Signal intensities of many stimuli mediated by lymphocyte receptors are regulated by the balance between activities of the receptor tyrosine kinase and tyrosine phosphatase. Lowered expression level of CD45 (RBBP4) mRNA would significantly affect immune functions. Expression levels of TAK1 (MAP3K7) located downstream of TGF-β or Toll-like receptors and an important signal-transducing molecule of these receptors, integrin α4 subunit, β1 integrin (ITGB1), and CD9 were also lowered, and mRNA expression levels were restored as the symptoms reached a state of remission. Thus, analysis of mRNA expression patterns of factors regulating functions of immune system cells was considered to be very useful for diagnosing depression.

Expression level of the heat shock protein (HSP) family that is induced by a variety of forms of environmental stress and that contributes to the acquisition of stress responsiveness and stress resistance of cells also showed relatively major variation in leukocytes of patients afflicted with depression. mRNA expression levels of MHC-associated HSP 70-1 (HSPA1A), HSPA 1B, HSPA 2, HSPA 6, HSPB 1, and HSPCA were elevated. Conversely, lowered expression levels of many heat shock proteins such as heat shock transcription factor (HSF2), DnaJ homolog (HSJ2), HSP105B, HSC70 (HSPA10), MHC class III HSP70 (HSPA4), mitochondrial HSP75 (HSPA9B), HSP27-1 (HSPB1), HSP90α (HSPCA), HSP90β (HSPCB), chaperonin HSP60 (HSPD1), and chaperonin 10 (HSPE1), the expression levels of which should be elevated by stress, were found as general features of patients afflicted with depression. This indicates that abnormality in normal mechanisms of stress resistance acquisition reflects clinical conditions. In particular, lowered expression levels of chaperonin HSP60 (HSPD1), HSP 70 (HSPA 10) that is constitutively expressed, and HSPA4 are observed commonly among patients afflicted with depression, and their expression levels are elevated as the symptoms reach a state of remission. These HSP families are considered to be a group of genes important for the diagnosis of depression. Further, variation in expression levels of stress-associated genes other than heat shock proteins was observed. The mRNA expression of a hypoxia-inducible factor (HIF1) that was a transcription factor serving as the key to hypoxia stress response or the glucocorticoid-responsive kinase (SGK) associated with the osmotic stress response was observed commonly among patients afflicted with depression, and the expression level of glucocorticoid a receptor was also lowered. These mRNA expression levels were restored along with recovery from the disease. Accordingly, environmental adaptability of patients afflicted with depression is lowered in general, and their fundamental cellular activities are lowered.

At present, mRNA expression levels of RNA polymerase II subunits or binding protein genes were both found to have been lowered, and their expression levels were found to have been restored as the disease state reached a state of remission, although association thereof with depression has not yet been clarified. Expression levels of a group of polymerase-associated genes, such as 140 kDa RNA polymerase II subunit protein gene (POLR2B), RNA polymerase II transcription elongation factor B (Sill) polypeptide 1 (TCEB1), RNA polymerase II transcription elongation factor B (SIII) polypeptide 1 homolog (TCEB1L), RNA polymerase II TATA box-binding protein-associated factor (AF2B), poly(A) polymerase, RNA polymerase β subunit, RNA polymerase III, UDP-galactose transporter novel isozyme (SLC35A1), and polymerase γ 2 accessory subunit (POLG2), reflected conditions of depression.

Recently, research into the causes of depression in relation to receptor signalings and transcription factors mediating distinct gene expressions has drawn attention, in addition to the search for association of metabolism of neurotransmitters including monoamine or receptors themselves with depression. A monoamine receptor is a G-protein-coupled receptor that activates inositol phosphate cycles and protein kinase C (PKC). This receptor also activates the elevation of cyclic AMP and the protein kinase A (PKA). Further, transcription factors activated by these signal transducing molecules and their gene products are focused, and it is expected that associations of these pathways with functional disorders will be discovered. Lithium derivatives, the effects of which as mood stabilizers for patients afflicted with bipolar disorders have been verified, are actually reported to act on signal-transducing pathways such as G-proteins, inositol phosphate cycles, PKC, PKA, glycogen synthase kinase 3-β, or Akt cascade, thereby exhibiting pharmacological actions (Br J Psychiatry 2001; 41: suppl 128-133).

Evidence that would support such reports was found in a group of genes associated with conditions of depression. Lowered mRNA expression levels of signal-transducing factors, such as PKCη (PRKCH), PKCnu (PRKCN), PKCβ1 isozyme, and phosphoinosidite 3′-kinase a subunit (PIK3CA), were observed. Lithium inactivates glycogen synthase kinase 3 and intensifies Wnt signals. In the case of patients afflicted with depression, expression levels of connective tissue growth factor-associated protein WISP-3, β-catenin (CTNNB1), transcription factor E2A (TCF3), and calmodulin-dependent calcineurin Aγ belonging to the Wnt signal pathway were lowered, and their expression levels were restored as the symptoms reached a state of remission. Lowered mRNA expression levels of GTP-binding proteins, i.e., RAB11A, RAB4, and RAB7L1, were observed, lowered expression levels of GTPase-activated protein rasp2l mRNA was observed, and their restoration through treatment was observed.

Concerning growth factor-associated proteins, mRNA expression levels of TGF-β-associated proteins, such as TGF-β receptor α, TGF-β-induced clone 22 homolog (TSC22), the aforementioned TGF-β signal transducing molecule TAK1, a member of the EGF receptor family ERBB4, and the insulin signal transducing molecule IRS4, reflected the symptoms of depression. In addition, mRNA expression levels of anti-oncogenes, i.e., Rb-associated protein RBBP7, growth inhibitory factors ING1, PTEN, and ST13, and oncogenes, such as RUNX1, KRAS2, MYCL1, and BMI1, were all lowered in patients afflicted with depression, and these expression levels were restored as the disease condition reached a state of remission. In a reflection of the expression patterns of these growth-associated genes, mRNA expression levels of CDC27, CDKN2C, CDK7, CCNB2, CCNG1, and PCNA associated with cell cycle were all lowered, and lowered mRNA expression levels of topoisomerase IIβ and topoisomerase II-binding protein (TOPBP 1) associated with DNA replication were observed. The evidence that suggests lowered general mitogen activity was deduced in leukocytes of patients afflicted with depression. Expression levels of these mRNA were also restored as the symptoms reached a state of remission. Lowered mRNA expression levels of the DNA repair enzyme MSH6, growth arrest-associated GADD45A, an apoptosis signal molecule DAP3 or API1, and caspase 10 were associated with symptoms of patients afflicted with depression. When variations in growth-associated genes were examined altogether, cell cycle was deduced to be generaly lowered in leukocytes of patients afflicted with depression.

In addition to genes belonging to other categories, such as ubiquitin-like proteins (UBL1), dioxin-responsible P450 (CYP1B1), and aldehyde dehydrogenase 1O, PTPRC, and CHST2 associated with the synthesis of leukocyte antigen Lewis X, and expression of transcription factors such as ARNTL or ELF2 also reflected the state of depression. This is, however, yet to be clarified.

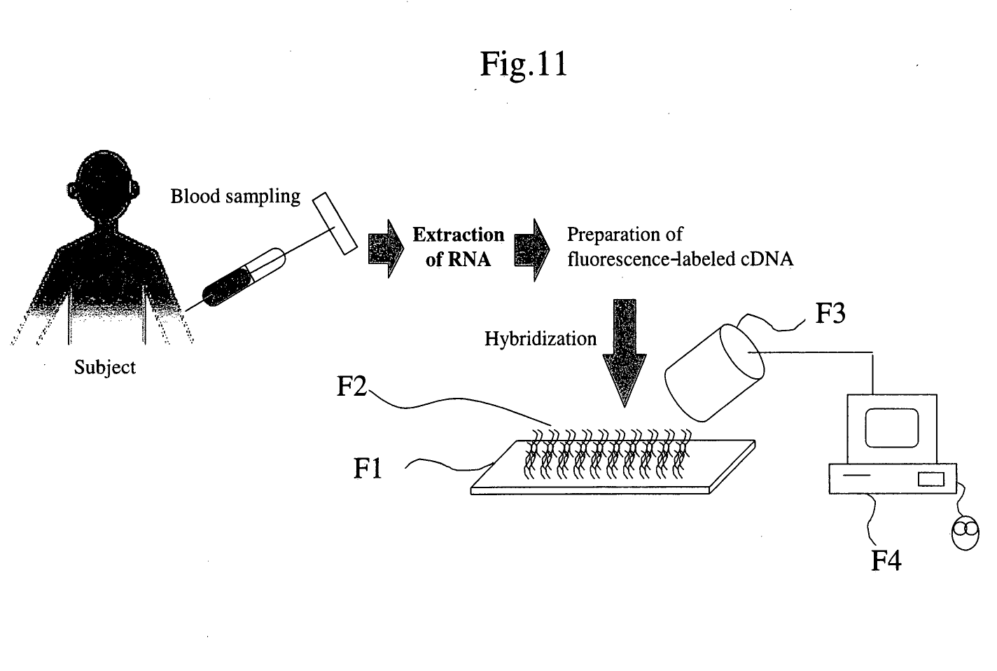

The present invention has been completed based on the results of above experimentation. FIG. 11 is a schematic representation of the present invention. In the present invention, peripheral blood is collected from. a subject, RNA is extracted, and its expression profile is examined, thereby resulting in diagnosis of depression in the subject. Approximately 2 to 5 ml of peripheral blood is sufficient for diagnosis.

Techniques for examining the gene expression levels employed in the present invention are not limited to DNA chips. For example, the availability of techniques such as nucleic acid hybridization utilizing other DNA-immobilized solid substrates such as DNA arrays or membrane filters, quantitative PCR such as RT-PCR or real-time PCR, Northern blotting, subtraction, differential display, differential hybridization, and cross-hybridization is apparent for those skilled in the art.

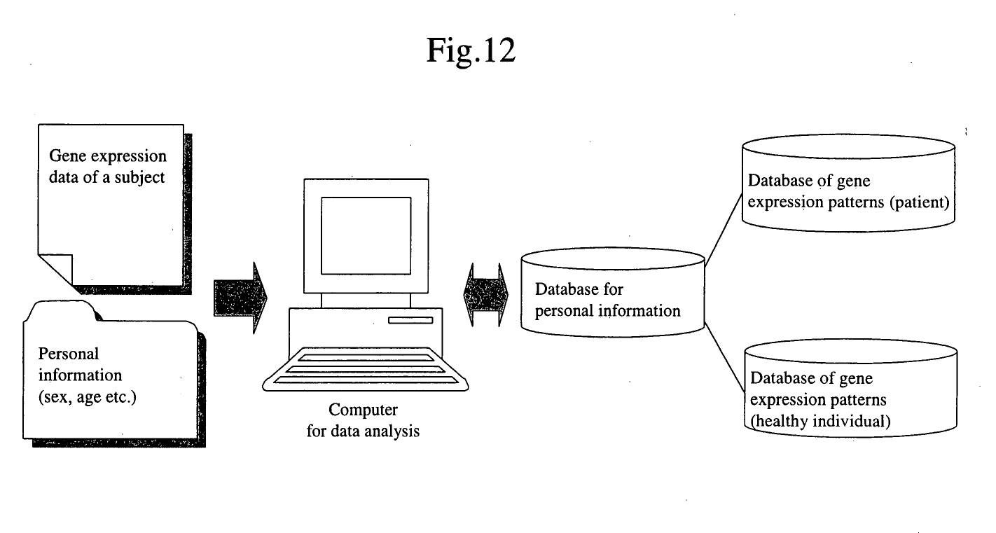

The RNA sample collected from a subject and the RNA sample collected from a healthy individual were respectively labeled with fluorescent dyes having different emission wavelengths, they were applied to the same DNA chip for diagnosing depression to conduct competitive hybridization, and the expression ratio of each gene between both samples can be determined as fluorescent intensity of each probe. Thus, the symptoms of depression in the subject can be evaluated. A certain RNA sample, for example, a commercialized universal RNA sample, is used as a control sample, and comparison and analysis of expression levels of the subject's sample and the control sample are conducted separately from those of the healthy individual's sample and the control sample to analyze expression data for both groups in comparison with each other. Thus, the conditions of depression in the subject can be evaluated. When comparing the sample of a healthy individual with that of the subject or their expression data, these two groups preferably have matching conditions in terms of age and sex. The conditions of depression in the subject are preferably evaluated by classifying the expression data for healthy individuals in accordance with their age and sex, storing them in a database, comparing the expression data for the subject with those for healthy individuals of the same age and sex, and analyzing the results of the comparison. A method for data analysis is not limited to clustering. A machine learning algorithm such as the one utilizing a support vector machine can be employed. Preferably, expression data for patients afflicted with depression and those for healthy individuals are previously stored in the computer, and the computer is allowed to determine which of the expression patterns for patients or healthy individuals are more similar to the subject's expression data, thereby diagnosing the conditions of depression in the subject. FIG. 12 schematically shows the system for diagnosing depression.

The present invention provides a method of diagnosing depression by collectively quantifying the RNA expression levels in peripheral blood to find evidence that is peculiar to depression. This would innovatively improve medical care for depression.

The method of the present invention can conduct the analysis with the use of 5 ml of blood obtained by conventional blood sampling without special cooperation provided by a patient. This diagnostic method can be carried out in a non-invasive, simple, and routine manner. This method of multidimensionally comprehending biological functions based on numerous mRNA expression levels is more adequate as a method of diagnosing complicated psychiatric diseases involving both mental and physical conditions such as depression in terms of its principle compared with the conventional method that assays only limited factors.

The results attained by the method of the present invention can be simply and clearly evaluated, they can be easily employed by primary care doctors as objective indicators for depression, and they are extremely useful for the establishment of diagnosis and introduction of therapy. A high-risk group can be simply, accurately, and cost-effectively selected from among the groups of people through medical checkups or complete physical examinations provided by workplaces, schools, and communities. This enables early detection of depression, and significantly contributes to the improvement of peoples' mental health from the viewpoint of preventive care.

The usefulness of the method according to the present invention is not limited to primary care and medical checkups. Specialists in psychiatric medicine can apply this technique to the search for psychological, social, and environmental factors associated with the development of depression, evaluation of clinical conditions, diagnosis, evaluation of treatment, and determination of prognosis. Thus, this technique can be a revolutionary test technique in the field of psychiatric medicine.

The present invention is hereafter described in greater detail with reference to the following examples, although it is not limited to these examples.

### EXAMPLE 1

Blood (5 ml each) was collected from healthy volunteers (12 in total; 2 males and 2 females each in their 30's, 40's, and 50's), and total RNA was extracted using a PAXgene Blood RNA System (Qiagen). The yield of total RNA was 5 μg to 15 μg. Subsequently, 5 μg each of total RNA extracted from each healthy volunteer was separated, and these samples were mixed with one another. An oligo (dT) 24 primer comprising a T7 promoter sequence added thereto was annealed to 5 μg of mixed total RNA, and first-strand DNA was synthesized. Thereafter, this first-strand DNA was used as a template to synthesize the second-strand DNA having a T7 promoter sequence. Finally, the second-strand DNA was used as a template to synthesize RNA by a T7 RNA polymerase. A random hexamer was allowed to anneal to 6 μg of the synthesized RNA to conduct a reverse transcription reaction, Cy3-dCTP was incorporated in the strand to synthesize fluorescence-labeled cDNA, and the resultant was determined to be a common control sample.

The target patients were as follows. Diagnosis was made in accordance with depressive episode specified in the International Classification of Diseases, 10th revision (ICD-10). Patients with serious physical complications or those taking therapeutic agents for physical diseases were excluded. 6 patients whose samples before treatment had been obtained were 3 males and 3 females aged 38 to 55 (44 years old on average), and their Hamilton scores were between 17 and 31 points (25.2 points on average). Blood was collected from patients (5 ml each), and total RNA was extracted using a PAXgene Blood RNA System (Qiagen). The yield of total RNA was 5 μg to 10 μg. Subsequently, an oligo (dT) 24 primer comprising a T7 promoter sequence added thereto was annealed to 5 μg of total RNA extracted from each patient, and first-strand DNA was synthesized. Thereafter, this first-strand DNA was used as a template to synthesize the second-strand DNA having a T7 promoter sequence. Finally, the second-strand DNA was used as a template to synthesize RNA with the aid of a T7 RNA polymerase. A random hexamer was allowed to anneal to 6 μg of RNA to conduct a reverse transcription reaction, and Cy5-dCTP was incorporated in the strand to synthesize fluorescence-labeled cDNA.

As a healthy control group, blood was collected from healthy volunteers (6 in total; one male and one female each in their 30's, 40's, and 50's), and Cy5-cDNA was synthesized in the same manner as in the case of the patients' samples.

Cy5-cDNA prepared from each patient's sample (6 μg) was mixed with the equivalent amount of Cy3-cDNA as a standard sample, the resultant was applied to a DNA chip (a DNA chip for analyzing drug response, Hitachi Co., Ltd.), and hybridization was carried out at 62° C. for 12 hours. After washing, fluorescence intensity at each spot was assayed using a scanner (ScanArray 5000, GSI-Lumonics), and the expression ratio of each gene between the standard sample and the patient's or healthy volunteer's sample can be determined as fluorescence intensity using quantifying software (QuantArray, GSI-Lumonics).

Group of genes having fluorescence intensities of 500 or higher in all data were selected as diagnostic markers. The ratios of expression intensities of the selected group of diagnostic marker genes (Table 1) are collectively shown in Table 2. As is apparent from Table 2, the expression levels of the diagnostic marker genes were significantly lowered in the group of patients afflicted with depression compared with those in the group of healthy volunteers.

Thus, diagnosis of depression through the analysis of the expression of specific group of genes is highly consistent with the results obtained through clinical findings. This indicates that the effectiveness of the present invention is very high.

TABLE 1
List of genes

| Symbol | Name | Category | GenBank |
|---|---|---|---|
| TCEB1L | transcription elongation factor B (SIII), polypeptide 1-like | polymerase, TF | Z47087 |
| COX7A2L | Homo sapiens cytochrome c oxidase subunit VIIa | mitcondria & | NM_004718 |
| | polypeptide 2 like | stress | |
| ACE | Homo sapiens dipeptidyl carboxypeptidase 1 (angiotensin I | angiotensin | NM_000789 |
| | converting enzyme) (ACE) | | |
| TOP2B | H. sapiens TOP2 mRNA for DNA topoisomerase II (partial).; | topoiosomerase | Z15115 |
| | Topoisomerase (DNA) II beta (180 kD) | | |
| ADAM17 | Homo sapiens snake venom-like protease (cSVP) mRNA, A | Cytokine | U92649 |
| | disintegrin and metalloproteinase domain 17 (tumor necrosis | | |
| | factor, alpha, converting enzyme) | | |
| BMI1 | Human prot-oncogene (BMI-1) mRNA, complete cds | oncogene | L13689 |
| POLR2B | polymerase (RNA) II (DNA directed) polypeptide B (140 kD) | polymerase | X63563 |
| PAP | poly(A) polymerase | polymerase | X76770 |
| PCNA | Homo sapiens proliferating cell nuclear antigen (PCNA) | CellCycle, | NM_002592 |
| | mRNA | Signal | |
| HSPD1 | Heat shock 60 kD protein 1 (chaperonin) | hsp | M34664 |
| TRAF5 | Homo sapiens mRNA for TRAF5, complete cds | Cytokine, | AB000509 |
| | | Signal | |
| RBBP7 | Human retinoblastoma-binding protein (RbAp46) mRNA, | Signal | U35143 |
| | complete cds | | |
| HSPA10 | Homo sapiens heat shock 70 kD protein 10 (HSC71) | hsp | NM_006597 |
| | (HSPA 10), mRNA | | |
| STAT4 | Homo sapiens STAT4 mRNA, complete cds | Signal, TF | L78440 |
| PRKCH | Human protein kinase C-L (PRKCL) mRNA; Protein kinase | Signal | M55284 |
| | C, eta | | |
| ST13 | Homo sapiens putative tumor suppressor ST13 (ST13) | Supressor | U17714 |
| | mRNA, complete cds | | |
| IKBKAP | Homo sapiens IkappaB kinase complex associated protein | Signal | AF044195 |
| | (IKAP) mRNA, complete cds; IKKAP2 | | |
| PRKCN | Homo sapiens EPK2 mRNA for serine/threonine kinase; | Signal | AB015982 |
| | Protein kinase C, nu | | |
| COX15 | Homo sapiens COX15 (yeast) homolog, cytochrome c | mitcondria & | NM_004376 |
| | oxidase assembly protein (COX15) | stress | |
| MSH6 | Human DNA mismatch repair protein MSH6; mutS alpha | DNA repair | U54777 |
| | 160-kDa subunit; G/T mismatch binding protein (GTMBP; | | |
| | GTBP) | | |
| ATP1B3 | ATPase, Na+/K+ transporting, beta 3 polypeptide | ATPase | U51478 |
| ATPIB3P1 | ATPase, Na+/K+ transporting, beta 3 pseudogene | ATPase | AF005898 |
| IL2RB | Human interleukin 2 receptor beta chain (p70-75) mRNA, | Cytokine, | M26062 |
| | complete cds | Signal | |
| CHST2 | Homo sapiens carbohydrate (N-acetylglucosamine-6-O) | sulfotransferase | NM_004267 |
| | sulfotransferase 2 (CHST2) | | |
| ATRX | Alpha thalassemia/mental retardation syndrome X-linked | ATPase | U72938 |
| API1 | Human inhibitor of apoptosis protein 2 mRNA; Apoptosis | Appoptosis, | U45879 |
| | inhibitor 1 | Signal | |
| CDC27 | Human homologue of S. pombe nuc2+ and A. nidulans | CellCycle | U00001 |
| | bimA; Cell division cycle 27 | | |
| ATP2C1 | ATPase, Ca++-sequestering | ATPase | AF225981 |
| PIK3CA | Human phosphoinositide 3′-hydroxykinase p110-alpha | Signal | U79143 |
| | subunit mRNA, Phosphoinositide-3-kinase, catalytic, alpha | | |
| | polypeptide | | |
| POLB | polymerase (DNA directed), beta | polymerase | D29013 |
| ITGA4 | Human integrin alpha 4 subunit mRNA, complete cds; | Signal | L12002 |
| | Integrin, alpha 4 (antigen CD49D, alpha 4 subunit of VLA-4 | | |
| | receptor) | | |
| CDKN2C | Homo sapiens cyclin-dependent kinase inhibitor (CDKN2C) | CellCycle | AF041248 |
| | mRNA, complete cds.; p18 | | |
| NRF | Homo sapiens transcription factor NRF | mitcondria & | NM_017544 |
| | | stress | |
| RAB11A | Homo sapiens rab11a GTPase mRNA, complete cds. | oncogene | AF000231 |
| SLC35A1 | solute carrier family 35 (CMP-sialic acid transporter), | polymerase | D87969 |
| | member 1 | | |
| UBL1 | ubiquitin-like 1 (sentrin) | Gap-junction | U61397 |
| TCEB1 | transcription elongation factor B (SIII), polypeptide 1 (15 kD, | polymerase, TF | L34587 |
| | elongin C) | | |
| HSPA4 | Human heat shock protein 70 (hsp70) mRNA; Heat shock | hsp | L12723 |
| | 70 kD protein 4 | | |
| WISP3 | Homo sapiens connective tissue growth factor related protein | Signal | AF100781 |
| | WISP-3 (WISP3) mRNA, complete cds. | | |
| IL16 | Homo sapiens putative IL-16 protein precursor, mRNA, | Cytokine | M90391 |
| | complete cds | | |
| ERBB4 | Homo sapiens receptor tyrosine kinase (ERBB4) gene, | oncogene | L07868 |
| | complete cds | | |
| MYCL1 | Human L-myc protein gene (MYCL1); gene only | oncogene | M19720 |
| CTNNB1 | H. sapiens mRNA for beta-catenin | Signal | X87838 |
| IL10RA | Human interleukin-10 receptor mRNA, complete cds | Cytokine | U00672 |
| DAP3 | Human ionizing radiation resistance conferring protein | Appoptosis | U18321 |
| | mRNA; Death associated protein 3 | | |
| GADD45A | Human growth arrest and DNA-damage-inducible protein | DNA-damage-inducible | M60974 |
| | (gadd45) mRNA | | |
| RPC39 | polymerase (RNA) III (DNA directed) (39 kD) | polymerase | U93869 |
| TCF3 | Human transcription factor (E2A) mRNA, complete cds | Signal, TF | M31523 |
| CDK7 | H. sapiens CDK activating kinase mRNA | CellCycle | X77743 |
| CASP10 | Human apoptotic cysteine protease Mch4 (Mch4) mRNA, | Appoptosis, | U60519 |
| | complete cds | Signal | |
| ING1 | Homo sapiens growth inhibitor p33ING1 (ING1) mRNA, | Signal, | AF001954 |
| | complete cds | Supressor | |
| RAB4 | Homo sapiens GTP-binding protein (RAB4) mRNA, | oncogene | M28211 |
| | complete cds. | | |
| IRF2 | Human mRNA for interferon regulatory factor-2 (IRF-2). | Cytokine | X15949 |
| TAF2B | TATA box binding protein (TBP)-associated factor, RNA | polymerase, TF | AF040701 |
| | polymerase II, B, 150 kD | | |
| CCNB2 | Human cyclin B2 mRNA, complete cds | CellCycle | AF002822 |
| CCNG1 | Human cyclin G1 mRNA, complete cds | CellCycle | U47413 |
| RAB7L1 | Homo sapiens mRNA for small GTP-binding protein, | oncogene | D84488 |
| | complete cds | | |
| PPP3CC | calcineurin A catalytic subunit [human, testis, mRNA, 2134 | Signal | S46622 |
| | nt]; Protein phosphatase 3 (formerly 2B), catalytic subunit, | | |
| | gamma isoform (calcineurin A gamma) | | |
| CCR5 | Human CC chemokine receptor 5 (CCR5) mRNA, complete | Signal | U54994 |
| | cds | | |
| CYP1B1 | Human dioxin-inducible cytochrome P450 (CYP1B1) | P450 | U03688 |
| | mRNA, complete cds | | |
| RUNX1 | Human AML1 mRNA for AML1b protein (alternatively | oncogene | D43968 |
| | spliced product),complete cds | | |
| SP100 | Human nuclear autoantigen (SP-100) mRNA | Signal | M60618 |
| TSC22 | Human putative regulatory protein TGF-beta-stimulated | GF | U35048 |
| | clone 22 homolog (TSC22) | | |
| CD9 | Homo sapiens CD9 antigen (p24) | hyperosmotic | NM_001769 |
| | | stress | |
| ELF2 | Human Ets transcription factor (NERF-2) mRNA, complete | TF | U43188 |
| | cds | | |
| PTEN | Human mutated in multiple advanced cancers protein | Supressor | U92436 |
| | (MMAC1) mRNA; putative protein-tyrosine phosphatase | | |
| | PTEN | | |
| SCYB5 | H. sapiens ENA-78 mRNA; Small inducible cytokine | Cytokine, | X78686 |
| | subfamily B (Cys-X-Cys), member 5 (epithelial-derived | Signal | |
| | neutrophil-activating peptide 78) | | |
| ADCY7 | Homo sapiens adenylate cyclase 7 (ADCY7) | Signal | NM_001114 |
| PRKCB1 | Human mRNA for protein kinase C (PKC) type beta I.; | Signal | X06318 |
| | Protein kinase C, beta 1 | | |
| ARNTL | Homo sapiens mRNA for BMAL1a; aryl hydrocarbon | Ah receptor | D89722 |
| | receptor nuclear translocator-like | | |
| TOPBP1 | Homo sapiens mRNA for DNA topoisomerase II binding | topoiosomerase | AB019397 |
| | protein, complete cds | | |
| IRS4 | Homo sapiens insulin receptor substrate 4 (IRS4) | Insulin | NM_003604 |
| TGFBR2 | Homo sapiens mRNA for TGF-betaIIR alpha, complete cds | GF, Signal | D50683 |
| RASA1 | Human GTPase-activating protein ras p21 (RASA) mRNA; | Signal | M23379 |
| | GAP | | |
| HIF1A | Homo sapiens hypoxia-inducible factor 1, alpha subunit | hypoxia, TF | NM_001530 |
| | (basic helix-loop-helix transcription factor) | | |
| NR3C1 | Human glucocorticoid receptor alpha mRNA, complete cds | glucocorticoids | M10901 |
| | | (Cortisol) | |
| KRAS2 | Human K-ras oncogene protein mRNA (KRAS2) | oncogene | M54968 |
| SGK | Homo sapiens serum/glucocorticoid regulated kinase | hyperosmotic | NM_005627 |
| | | stress | |
| MAP3K7 | Homo sapiens mitogen-activated protein kinase kinase kinase | Signal | NM_003188 |
| | 7 (MAP3K7), mRNA, TAK1 | | |
| POLG2 | polymerase (DNA directed), gamma 2, accessory subunit | polymerase | U94703 |
| ALDH10 | Human microsomal aldehyde dehydrogenase (ALD10) | ALDH | U46689 |
| | mRNA | | |
| RBBP4 | Human chromatin assembly factor 1 p48 subunit (CAF1 p48 | Signal | X74262 |
| | subunit); retinoblastoma-binding protein 4 | | |
| ITGB1 | Integrin, beta 1 (fibronectin receptor, beta polypeptide, | Signal | X07979 |
| | antigen CD29 includes MDF2, MSK12); | | |
| PTPRC | Human mRNA for T200 leukocyte common antigen (CD45, | Signal | Y00062 |
| | LC-A). | | |

TABLE 2
Logarithms (base: 2) of the ratios of expression intensities obtained in Examples

| | Group of patients | | | | | | Group of healthy controls | | | | | |
|---|---|---|---|---|---|---|---|---|---|---|---|---|
| | Sample No. | | | | | | | | | | | |
| | No. 1 | No. 2 | No. 3 | No. 4 | No. 5 | No. 6 | No. 7 | No. 8 | No. 9 | No. 10 | No. 11 | No. 12 |
| | Age/Sex | | | | | | | | | | | |
| | 45/male | 38/male | 39/female | 55/male | 41/female | 46/male | 33/male | 47/male | 42/female | 38/female | 53/female | 52/male |
| Gene | Hamilton Score | | | | | | | | | | | |
| symbol | 29 pts. | 23 pts. | 23 pts. | 17 pts. | 28 pts. | 31 pts. | | | | | | |
| TCEB1L | −1.22 | 0.16 | −0.18 | −0.20 | 0.24 | −0.98 | 0.98 | −0.06 | 0.07 | 0.28 | 0.26 | 0.38 |
| COX7A2L | −1.57 | −0.07 | 0.03 | −0.26 | −0.19 | −1.28 | −0.03 | 0.32 | 0.18 | 0.30 | 0.24 | 0.48 |
| ACE | −1.09 | 0.27 | −0.05 | −0.04 | −0.01 | −1.00 | 0.33 | 0.82 | 0.42 | 0.03 | 0.13 | 0.25 |
| TOP2B | −1.41 | −0.22 | −0.61 | 0.00 | −0.56 | −1.63 | −0.13 | −0.35 | −0.14 | −0.62 | −0.11 | 0.13 |
| ADAM17 | −1.28 | −0.07 | −0.58 | −0.08 | −0.40 | −1.34 | −0.13 | −0.49 | 0.07 | 0.20 | 0.06 | −0.03 |
| BMI1 | −0.96 | −0.16 | −0.12 | −0.26 | −0.74 | −1.34 | 0.36 | −0.17 | 0.07 | 0.26 | −0.09 | 0.45 |
| POLR2B | −0.97 | 0.07 | −0.24 | 0.15 | −0.50 | −1.44 | 0.14 | −0.05 | 0.04 | 0.15 | 0.40 | 0.35 |
| PAP | −0.80 | 0.06 | −0.39 | 0.05 | −0.47 | −1.27 | −0.08 | 0.43 | 0.05 | 0.15 | 0.23 | 0.11 |
| PCNA | −1.29 | 0.13 | −0.26 | −0.19 | −0.71 | −1.58 | −0.21 | −0.38 | 0.24 | 0.24 | 0.51 | 0.33 |
| HSPD1 | −1.15 | −0.13 | −0.28 | 0.07 | −0.40 | −1.58 | −0.49 | −0.35 | −0.16 | 0.13 | 0.19 | −0.02 |
| TRAF5 | −0.75 | 0.08 | −0.20 | −0.35 | 0.04 | −1.81 | 0.35 | −0.01 | 0.01 | 0.44 | 0.04 | 0.22 |
| RBBP7 | −0.86 | 0.34 | −0.15 | −0.39 | 0.24 | −1.49 | 0.25 | −0.61 | 0.24 | 0.50 | 0.36 | 0.29 |
| HSPA10 | −0.71 | 0.25 | −0.37 | −0.36 | −0.17 | −1.49 | 0.19 | 0.52 | 0.12 | 0.16 | 0.31 | 0.13 |
| STAT4 | −0.37 | −0.25 | −0.62 | −0.65 | −0.28 | −1.54 | 0.51 | 1.17 | −0.16 | −0.13 | 0.04 | 0.54 |
| PRKCH | −0.81 | −0.05 | −0.07 | −0.47 | −0.32 | −1.29 | 0.45 | 0.64 | 0.38 | 0.24 | 0.39 | 0.45 |
| ST13 | −0.77 | −0.16 | −0.08 | −0.19 | 0.06 | −1.35 | 0.29 | 0.89 | 0.18 | 0.39 | 0.13 | 0.52 |
| IKBKAP | −0.51 | −0.18 | −0.28 | −0.28 | 0.04 | −1.44 | 0.02 | −0.22 | −0.03 | 0.47 | 0.02 | −0.18 |
| PRKCN | −0.99 | −0.31 | 0.19 | −0.28 | −0.22 | −0.66 | 0.22 | 1.31 | 0.02 | 0.16 | −0.09 | 0.45 |
| COX15 | −0.56 | −0.17 | −0.22 | −0.43 | −0.24 | −0.88 | −0.11 | 0.22 | 0.47 | 0.54 | 0.42 | 0.23 |
| MSH6 | −0.47 | −0.11 | −0.40 | −0.22 | −0.67 | −1.24 | 0.23 | −0.33 | 0.29 | 0.31 | 0.19 | 0.21 |
| ATP1B3 | −1.14 | 0.02 | 0.23 | −0.39 | 0.05 | −1.04 | −0.13 | 0.01 | 0.13 | 0.49 | 0.49 | 0.34 |
| ATP1B3P1 | −0.82 | −0.11 | 0.09 | −0.57 | −0.09 | −1.07 | −0.02 | 0.35 | 0.24 | 0.29 | 0.57 | 0.39 |
| IL2RB | −0.65 | −0.21 | 0.23 | −0.24 | −0.01 | −0.73 | 0.12 | −0.14 | 0.13 | 0.38 | 0.30 | −0.02 |
| CHST2 | −1.08 | −0.01 | 0.11 | −0.30 | −0.08 | −1.15 | −0.15 | −0.42 | 0.51 | 0.54 | 0.65 | 0.00 |
| ATRX | −1.06 | 0.09 | −0.24 | −0.11 | 0.08 | −1.12 | 0.02 | −0.68 | 0.27 | 0.17 | 0.07 | −0.11 |
| API1 | −0.62 | −0.06 | −0.75 | 0.14 | −0.61 | −1.02 | 0.42 | 0.69 | −0.03 | 0.16 | 0.03 | −0.44 |
| CDC27 | −0.21 | 0.17 | −0.41 | −0.05 | −0.21 | −0.87 | 0.33 | 0.96 | 0.29 | 0.15 | 0.49 | 0.43 |
| ATP2C1 | −0.46 | −0.10 | −0.19 | 0.07 | −0.30 | −1.08 | 0.39 | 0.75 | 0.25 | 0.17 | 0.31 | 0.37 |
| PIK3CA | −0.49 | −0.03 | −0.17 | 0.05 | −0.82 | −0.71 | 0.44 | 0.72 | 0.17 | 0.28 | 0.24 | 0.09 |
| POLB | −0.07 | −0.19 | −0.52 | 0.00 | −0.41 | −0.92 | 0.21 | 0.82 | 0.19 | −0.17 | 0.17 | 0.04 |
| ITGA4 | −0.20 | −0.63 | −0.90 | −0.25 | −0.62 | −1.16 | 0.69 | 1.38 | 0.44 | 0.29 | 0.67 | 0.44 |
| CDKN2C | −0.98 | −0.15 | −0.01 | −0.13 | −0.01 | −0.34 | −0.05 | −0.25 | 0.10 | 0.33 | 0.20 | 0.20 |
| NRF | −0.88 | 0.15 | −0.51 | −0.22 | −0.06 | −0.82 | −0.04 | −0.29 | 0.35 | 0.33 | 0.25 | 0.04 |
| RAB11A | −0.80 | 0.08 | 0.10 | 0.11 | −0.16 | −0.55 | 0.05 | −0.29 | 0.44 | 0.25 | 0.26 | 0.43 |
| SLC35A1 | −0.33 | −0.18 | −0.18 | 0.03 | 0.07 | −0.29 | 0.47 | 0.96 | 0.32 | 0.35 | 0.22 | 0.75 |
| UBL1 | −0.67 | 0.17 | −0.25 | 0.03 | −0.27 | −0.83 | 0.11 | 1.11 | 0.13 | 0.13 | 0.04 | 0.60 |
| TCEB1 | −0.55 | −0.10 | −0.09 | −0.21 | −0.16 | −0.60 | −0.08 | 0.47 | 0.32 | 0.57 | 0.50 | 0.20 |
| HSPA4 | −1.07 | −0.10 | −0.27 | −0.35 | 0.14 | −1.39 | −0.84 | 0.00 | −0.13 | −0.03 | 0.19 | 0.47 |
| WISP3 | −0.56 | 0.03 | −0.09 | −0.17 | −0.22 | −1.08 | 1.00 | 0.67 | 0.24 | 0.19 | 0.12 | 0.51 |
| IL16 | −0.14 | −0.01 | 0.03 | −0.13 | 0.21 | −0.57 | 0.14 | 0.07 | 0.13 | 0.00 | 0.18 | 0.65 |
| ERBB4 | 0.02 | 0.03 | 0.25 | −0.19 | 0.07 | −0.51 | 0.00 | 0.79 | 0.28 | 0.01 | 0.38 | 0.23 |
| MYCL1 | −0.20 | −0.13 | 0.47 | −0.13 | 0.22 | −0.35 | 0.07 | 0.55 | 0.26 | 0.32 | 0.24 | 0.31 |
| CTNNB1 | −0.21 | 0.03 | 0.28 | 0.12 | 0.05 | −0.73 | 0.31 | 1.52 | 0.33 | 0.31 | 0.16 | −0.31 |
| IL10RA | −0.20 | 0.19 | 0.29 | 0.20 | 0.33 | −1.07 | 0.39 | −0.21 | 0.46 | 0.42 | 0.38 | 0.14 |
| DAP3 | −0.25 | 0.26 | 0.00 | −0.21 | 0.35 | −0.87 | 0.02 | 0.40 | 0.44 | 0.32 | 0.38 | 0.28 |
| GADD45A | −0.32 | 0.18 | −0.16 | 0.16 | 0.08 | −1.04 | −0.12 | 0.36 | 0.40 | 0.40 | 0.17 | 0.33 |
| RPC39 | −0.37 | 0.18 | −0.13 | 0.21 | 0.10 | −1.06 | −0.05 | −0.01 | 0.42 | 0.43 | 0.15 | 0.21 |
| TCF3 | −0.01 | 0.13 | 0.01 | −0.18 | 0.08 | −0.93 | −0.36 | 0.33 | 0.44 | 1.10 | 0.46 | 0.35 |
| CDK7 | 0.24 | −0.23 | −0.15 | 0.17 | −0.29 | −0.52 | 0.06 | 0.66 | 0.05 | 0.16 | 0.29 | 0.45 |
| CASP10 | 0.44 | −0.12 | 0.04 | 0.06 | −0.06 | −0.65 | −0.15 | 0.88 | 0.35 | 0.62 | 0.53 | 0.50 |
| ING1 | 0.02 | −0.15 | 0.07 | −0.07 | −0.21 | −0.88 | 0.13 | 0.20 | 0.33 | 0.40 | 0.24 | 0.37 |
| RAB4 | −0.42 | −0.14 | 0.22 | −0.17 | −0.13 | −0.42 | 0.35 | 0.92 | 0.39 | −0.73 | 0.25 | 0.74 |
| IRF2 | 0.20 | −0.31 | 0.08 | −0.09 | −0.53 | −0.13 | 0.02 | 0.73 | 0.11 | 0.24 | 0.27 | 0.11 |
| TAF2B | −0.92 | −0.32 | 0.37 | −0.26 | −0.35 | −0.95 | 0.31 | 0.14 | 0.11 | 0.42 | −0.03 | 0.59 |
| CCNB2 | −0.85 | −0.29 | 0.63 | −0.23 | −0.20 | −0.84 | 0.17 | 0.14 | 0.37 | −0.04 | 0.18 | 0.68 |
| CCNG1 | −0.99 | −0.06 | −0.60 | −0.11 | −0.78 | −0.58 | 0.28 | 0.89 | 0.12 | 0.05 | 0.25 | 0.20 |
| RAB7L1 | −0.64 | −0.41 | 0.18 | −0.39 | −0.12 | −0.49 | 0.25 | 1.01 | 0.05 | 0.23 | 0.48 | 0.57 |
| PPP3CC | −1.13 | −0.06 | −0.13 | −0.28 | −0.22 | 0.13 | −0.04 | −0.74 | 0.27 | −0.09 | 0.24 | 0.40 |
| CCR5 | −0.53 | 0.08 | 0.59 | −0.96 | −0.05 | −1.11 | 0.20 | 0.34 | 0.14 | −0.14 | 0.40 | −0.25 |
| CYP1B1 | 0.17 | 0.41 | 0.61 | 0.48 | 0.15 | −1.00 | 0.16 | −0.14 | 0.49 | 0.35 | 0.86 | 0.35 |
| RUNX1 | 0.40 | 0.35 | 0.37 | −0.07 | −0.18 | −1.04 | −0.06 | 1.35 | 0.19 | −1.18 | 0.20 | 0.03 |
| SP100 | 0.46 | 0.25 | −0.62 | 0.19 | 0.01 | −0.93 | 0.29 | −0.43 | 0.27 | 0.39 | 0.43 | 0.29 |
| TSC22 | −1.10 | −1.06 | −0.57 | −0.21 | 0.32 | −1.16 | 0.30 | 0.22 | 0.90 | 0.21 | −0.32 | 0.37 |
| CD9 | −0.41 | −0.42 | −0.35 | −0.75 | 0.00 | −0.17 | 1.51 | 2.34 | 0.24 | 0.36 | 0.18 | 0.68 |
| ELF2 | −0.52 | −0.73 | −0.35 | −0.08 | −0.74 | −1.26 | 0.17 | −0.11 | −0.17 | 0.03 | −0.18 | 0.24 |
| PTEN | 0.06 | −0.43 | −0.25 | 0.50 | −0.80 | −1.66 | 0.36 | 0.28 | 0.25 | 0.47 | 0.18 | 0.53 |
| SCYB5 | 0.20 | −0.37 | 0.03 | 0.50 | −0.46 | −1.29 | 0.04 | −0.25 | 0.36 | −0.14 | −0.22 | −0.05 |
| ADCY7 | 0.26 | −0.37 | −0.17 | 0.06 | −0.63 | −1.20 | 0.22 | 0.65 | −0.03 | 0.20 | 0.07 | 0.10 |
| PRKCB1 | 0.07 | −0.46 | 0.06 | 0.32 | −0.81 | −1.04 | 0.15 | −0.81 | −0.02 | −0.04 | −0.03 | 0.11 |
| ARNTL | 0.07 | −0.43 | 0.14 | 0.14 | −0.87 | −1.53 | 0.10 | −0.34 | 0.05 | −0.35 | −0.12 | 0.29 |
| TOPBP1 | −0.18 | −0.17 | −0.23 | 0.02 | −0.53 | −1.64 | 0.03 | −0.48 | 0.06 | 0.18 | 0.06 | 0.14 |
| IRS4 | 0.15 | −0.55 | −0.11 | 0.21 | −0.32 | −1.72 | −0.02 | −1.02 | −0.19 | −0.02 | −0.32 | −0.04 |
| TGFBR2 | −0.13 | −0.53 | −0.28 | 0.13 | −0.86 | −2.12 | 0.49 | 0.00 | 0.06 | −0.22 | −0.26 | 0.36 |
| RASA1 | −0.04 | −0.15 | −0.16 | −0.02 | −0.90 | −2.14 | 0.35 | 0.23 | 0.32 | 0.36 | 0.30 | 0.60 |
| HIF1A | 0.37 | −0.08 | −0.46 | 0.34 | −0.91 | −1.57 | 0.27 | 0.21 | 0.47 | 0.20 | −0.05 | 0.04 |
| NR3C1 | −0.64 | −0.48 | −0.16 | 0.33 | −0.79 | −1.82 | 0.31 | 0.47 | 0.17 | 0.13 | 0.16 | 0.35 |
| KRAS2 | −1.17 | −0.40 | −0.17 | −0.03 | −0.84 | −1.68 | −0.07 | −0.50 | 0.13 | 0.52 | 0.18 | 0.30 |
| SGK | −0.66 | −0.46 | −0.03 | −0.14 | −0.69 | −1.94 | 0.12 | −0.81 | −0.24 | 0.21 | −0.36 | −0.40 |
| MAP3K7 | −1.05 | −0.27 | −0.51 | −0.33 | −0.88 | −2.35 | 0.01 | −0.76 | 0.25 | 0.03 | 0.04 | 0.22 |
| POLG2 | 0.02 | 0.04 | −0.10 | −0.07 | −0.25 | −1.47 | 0.34 | 0.27 | 0.17 | 0.52 | 0.07 | 0.28 |
| ALDH10 | −0.28 | −0.66 | 0.34 | −0.39 | −0.34 | −1.22 | 0.19 | 0.22 | 0.24 | 0.71 | 0.44 | 0.61 |
| RBBP4 | −0.26 | −0.04 | −0.09 | −0.11 | −0.21 | −1.50 | 0.46 | 1.32 | 0.58 | 0.46 | 0.57 | 0.95 |
| ITGB1 | 0.10 | −0.51 | −0.56 | 0.11 | −0.67 | −2.07 | 0.65 | 0.61 | 0.14 | 0.09 | −0.03 | 0.81 |
| PTPRC | −0.53 | −0.28 | −0.53 | 0.08 | −1.38 | −2.47 | 0.41 | −0.61 | −0.02 | −0.01 | 0.11 | 0.28 |
| Mean | −0.50 | −0.13 | −0.13 | −0.10 | −0.29 | −1.13 | 0.18 | 0.25 | 0.20 | 0.20 | 0.20 | 0.29 |

The present invention has been completed based on the results of experimentation in which the gene expression in the peripheral blood leukocytes of patients afflicted with depression had been analyzed. The utilization of the diagnostic method of the present invention enables the simple and accurate diagnosis of depression.

All publications, patents, and patent applications cited herein are incorporated herein by reference in their entirety.

1. A method of diagnosing depression, wherein the expression level of at least one gene selected from among apoptosis-associated genes, ATPase-associated genes, cell cycle-associated genes, cytokine-associated genes, heat shock protein-associated genes, polymerase-associated genes, GTP-binding protein-associated genes, protein kinase C-associated genes, and mitochondrial cytochrome C oxidase-associated genes is analyzed using mRNA of a subject's peripheral blood, and symptoms of the subject are diagnosed based on the results of analysis.

2. A method of diagnosing depression, wherein the expression level of at least one gene selected from among the genes listed in Table 1 is analyzed using mRNA of a subject's peripheral blood, and symptoms of the subject are diagnosed based on the results of such analysis.

3. The method of diagnosing depression according to claim 1, wherein symptoms of a subject are diagnosed by analyzing gene expression profiles.

4. The method of diagnosing depression according to claim 1, wherein symptoms of a subject are diagnosed by analyzing expression profiles of the genes listed in Table 1.

5. The method according to claim 1, wherein symptoms of a subject are diagnosed by analyzing gene expression profiles with the cluster analysis technique.

6. The method of diagnosing depression according to claim 1, wherein the gene expression profiles of a subject and that of a healthy individual are compared, and the results of the comparison are analyzed, thereby allowing diagnosis regarding symptoms of the subject.

7. The method of diagnosing depression according to claim 6, wherein data concerning age and sex, in addition to gene expression profiles, is compared and analyzed.

8. The method of diagnosing depression according to claim 1, wherein the gene expression profiles of a single subject before and after treatment are compared, and the results of the comparison are analyzed, thereby allowing diagnosis regarding symptoms of the subject.

9. A method of diagnosing depression, wherein the gene expression level of at least one gene selected from among the genes listed in Table 1 is analyzed using mRNA of a subject's peripheral blood, the gene expression profiles of the subject is compared with that of a healthy individual, and the results of the comparison are analyzed, thereby allowing diagnosis regarding symptoms of the subject.

10. The method of diagnosing depression according to claim 9, wherein data concerning age and sex, in addition to gene expression profiles, is compared and analyzed.

11. A method of diagnosing depression, wherein the gene expression level of at least one gene selected from among the genes listed in Table 1 is analyzed using mRNA of a subject's peripheral blood, the gene expression profiles of a single subject before and after treatment are compared, and the results of the comparison are analyzed, thereby allowing diagnosis regarding symptoms.

12. The method of diagnosing depression according to claim 1, wherein the gene expression levels are analyzed utilizing DNA chips.

13. The method of diagnosing depression according to claim 1, wherein mRNA derived from peripheral blood is derived from peripheral leukocytes.

14. A solid substrate for diagnosing depression comprising a probe, immobilized on the substrate, that specifically hybridizes to at least one gene selected from among the genes listed in Table 1 to detect the selected gene.

15. A system for diagnosing depression utilizing the method of diagnosing depression according to claim 1, wherein the data concerning a subject, such as gene expression level, age, and sex, are compared with data that had been previously acquired concerning a healthy individual, and the results of the comparison are analyzed.
